SEQUENCE LISTING (9 sequences)

SEQ ID NO: 1 - protein, 319 aa, Homo sapiens
MSQQNTSGDCLFDGVNELMKTLQFAVHIPTFVLGLLLNLLAIHGFSTFLKNRWPDYAATSIYMINLAVFDLLLVLSLPFKMVLSQVQSPFPSLCTLVECLYFVSMYGSVFTICFISMDRFLAIRYPLLVSHLRSPRKIFGICCTIWVLVWTGSIPIYSFHGKVEKYMCFHNMSDDTWSAKVFFPLEVFGFLLPMGIMGFCCSRSIHILLGRRDHTQDWVQQKACIYSIAASLAVFVVSFLPVHLGFFLQFLVRNSFIVECRAKQSISFFLQLSMCFSNVNCCLDVFCYYFVIKEFRMNIRAHRPSRVQLVLQDTTISRG

SEQ ID NO: 2 - protein, 319 aa, Homo sapiens
MSQQNTSGDCLFDGVNELMKTLQFAVHIPTFVLGLLLNLLAIHGFSTFLKNRWPDYAATSIYMINLAVFDLLLVLSLPFKMVLSQVQSPFPSLCTLVECLYFVSMYGSVFTICFISMDRFLAIRYPLLVSHSGPPGRSLGSACTIWVLVWTGSIPIYSFHGKVEKYMCFHNMSDDTWSAKVFFPLEVFGFLLPMGIMGFCCSRSIHILLGRRDHTQDWVQQKACIYSIAASLAVFVVSFLPVHLGFFLQFLVRNSFIVECRAKQSISFFLQLSMCFSNVNCCLDVFCYYFVIKEFRMNIRAHRPSRVQLVLQDTTISRG

SEQ ID NO: 3 - DNA, 27 bp, Artificial
  Oligonucleotide Primer
atgagtcagcaaaacaccagtggggac

SEQ ID NO: 4 - DNA, 24 bp, Artificial
  Oligonucleotide Primer
ttagccccgggagatcgtggtgtc

SEQ ID NO: 5 - DNA, 69 bp, Artificial
  Oligonucleotide Primer
caagctagctatgccaccatggactacaaggacgacgatgacaagagtcagcaaaacaccagtggggac

SEQ ID NO: 6 - DNA, 36 bp, Artificial
  Oligonucleotide Primer
cctggatccaaattagccccgggagatcgtggtgtc

SEQ ID NO: 7 - protein, 11 aa, Homo sapiens
LRSPRKIFGIC

SEQ ID NO: 8 - protein, 11 aa, Homo sapiens
SGPPGRSLGSA

SEQ ID NO: 9 - DNA, 960 bp, Homo sapiens
atgagtcagcaaaacaccagtggggactgcctgtttgacggtgtcaacgagctgatgaaaaccctacagtttgcagtccacatccccaccttcgtcctgggcctgctcctcaacctgctggccatccatggcttcagcaccttccttaagaacaggtggcccgattatgctgccacctccatctacatgatcaacctggcagtctttgacctgctgctggtgctctccctcccattcaagatggtcctgtcccaggtacagtcccccttcccgtccctgtgcaccctggtggagtgcctttacttcgtcagcatgtacggaagcgtcttcaccatctgcttcatcagcatggaccggttcttggccatccgttacccgctactggtgagccactccggtcccccaggaagatctttgggatctgcatgcacaatctgggtcctggtgtggaccggaagcatccctatctacagtttccatgggaaagtggaaaaatacatgtgcttccacaacatgtctgatgatacctggagcgccaaggtcttcttcccgctggaggtgtttggcttcctccttcccatgggcatcatgggcttctgctgctccaggagcatccacatcctgctgggccgccgagaccacacccaggactgggtgcagcagaaagcctgcatctacagcatcgcagccagcctggctgtattcgtggtctccttcctcccagtccacctggggttcttcctgcagttcctggtgagaaacagctttatcgtagagtgcagagccaagcagagcatcagcttcttcttgcaattgtccatgtgtttctccaatgtcaactgctgcctggatgttttctgctactactttgtcatcaaagaattccgcatgaacatcagggcccaccggccttccagggtccagctggtcctgcaggacaccacgatctcccggggctaa

(19) United States
(12) Patent Application Publication
(10) Pub. No.: US 2006/0194260 A1
(43) Pub. Date: Aug. 31, 2006

(54) Screening assays for cannabinoid-ligand-type modulators of gpr55

(75) Inventors: Tomas Drmota, Molndal (SE); Peter Greasley, Molndal (SE); Thierry Groblewski, Montreal (CA)

Correspondence Address: ASTRAZENECA R&D BOSTON, 35 GATEHOUSE DRIVE, WALTHAM, MA, 02451-1215, US

(73) Assignee: AstraZeneca AB, Sodertaije (SE)

(21) Appl. No.: 10/545,851

(22) PCT Filed: Feb. 13, 2004
(86) PCT No.: PCT/GB04/00571

§ 371 Date: Aug. 16, 2005

Related U.S. Application Data
(60) Provisional application No. 60/448,665, filed on Feb. 18, 2003.

(51) Int. Cl. G01N 33/567; G01N 33/53

(52) U.S. Cl. 435/7.2

(57) ABSTRACT

Screening assays for compounds that modulate the interaction between cannabinoids and the GPR55 receptor are disclosed. Furthermore, a method for determining the selectivity of a test compound against a panel of cannabinoid receptors including GPR55 is disclosed.

### FIELD OF THE INVENTION

The present invention relates to screening assays for compounds that modulate the interaction between cannabinoids and other ligands and the GPR55 receptor.

### BACKGROUND

Preparations of Cannabis sativa have been used for medicinal and recreational purposes for at least 4,000 years. Recently, cannabinoids have been the subject of renewed interest for their potential medicinal applications, e.g., in analgesia, nausea and appetite stimulation.

Cannabanoids exert their effects by binding to specific receptors located in the cell membrane. Two types of high-affinity cannabinoid receptors have been identified to date by molecular cloning: 1) CB1 receptors (Devane et al., 1988, Mol. Pharmacol., 34:605-613; Matsuda et al., 1990, Nature, 346:561-564; Shire et al., 1995, J. Biol. Chem., 270:3726-3731; Ishac et al., 1996, Br. J. Pharmacol., 118:2023-2028), and 2) CB2 receptors (Munro et al., 1993, Nature, 365:61-65). Both CB1 and CB2 are coupled to the Gᵢ, G-protein signal transduction pathway. Activation of the cannabinoid receptors leads to inhibition of adenylate cyclase and activation of MAP kinase. CB1 receptors can also modulate ion channels, inhibiting N-, and P/R-type calcium channels, stimulating inwardly rectifying K⁺ channels and enhancing the activation of the A-type K⁺ channel.

CB1 receptors are primarily, but not exclusively, expressed in the CNS and are believed to mediate the CNS effects of endogenous (e.g., anandamide) and exogenously applied cannabinoids. Peripheral areas of expression include, but are not restricted to, the pituitary gland, immune cells, reproductive tissues, gastrointestinal tissues, superior cervical ganglion, heart, lung, urinary bladder, and adrenal gland. CB1 receptors are also located on central and peripheral nerve terminals and, when activated, seem to suppress the neuronal release of a number of excitatory and inhibitory transmitters including acetylcholine, noradrenaline, dopamine, 5 hydroxy-tryptamine, γ-aminobutyric acid, glutamate and aspartate (Pertwee, 1997, Pharmacol. Ther., 129:74; Ong & Mackie, 1999, Neuroscience, 92:1177; Pertwee, 2001, Progr. Neurobiol., 63:569). CB2 receptor expression is, however, restricted to the periphery, mainly in immune cells with particularly high levels in B-cells and natural killer cells (Galiégue et al., 1995, Eur. J. Biochem, 54:232).

A number of endogenous ligands have been identified which are believed to modulate the cannabinoid system via the previously identified CB1 and CB2 receptors, or by their action at as yet unidentified receptors. These ligands include, but are not limited to, anandamide, 2-aracadonylglycerol, noladin ether, palmitoylethanolamine, virohdaine and oleylthanolamide.

Endocannabinoids have been shown to play a role in the physiological regulation of food intake and body weight. Cannabinoid agonists are known to enhance appetite (Fride, 2002, Prostaglandins Leukot. Essent. Fatty Acids, 66:221-233). Studies have shown that anandamide increases food intake in rats, while the non-endogenous antagonist SR141716 (Sanofi Research) inhibits the in-take of palatable food.

Studies suggest that endocannabinoids regulate multiple physiological and pathological reproductive functions (Maccarrone et al., 2002, Prostaglandins Leukot. Essent. Fatty Acids, 66:309-317). The levels of uterine anandamide have been shown to affect pregnancy and embryo development in mice. High levels of anandamide in the mouse uterus have been associated with a decrease in uterine receptivity. Additionally, in women experiencing miscarriage, blood levels of anandamide were 4-fold higher than the levels in women with normal gestation.

Recent studies suggest that endocannabinoids, such as 2-arachidonoylglycerol, play a role in the progression of the pathophysiology of shock, since a selective CB1 antagonist reverses the hemorrhagic-induced shock condition (Cainazzo, 2002, Eur. J. Pharmacol., 441:91-97).

Endocannabinoids have also been shown to act as immunomodulators, generally exerting a negative action on the onset of a variety of parameters of the immune response (Parolaro et al, 2002, Prostaglandins Leukot. Essent. Fatty Acids, 66:319-32). Previous studies have shown that the CB2 receptor plays a very important role in the stimulation of growth of several, if not all, hematopoietic lineages (Valk et al., 1997, Blood, 90:1448-1457; Derocq, 2000, J. Biol. Chem, 275:15621-15628).

Thus, cannabinoid receptors are physiologically or pathophysiologically relevant in a great diversity of therapeutic areas, encompassing symptoms, disorders or syndromes of the central nervous, cardiovascular, endocrine or gastrointestinal systems. Specifically, the involvement of CB1 receptors has been implicated in altered pain perception, cognition and memory, addiction/substance abuse, schizophrenia/psychosis/delusion disorders, psychological, neurological, neurodegenerative (i.e., Parkinson's disease), stress, mood modulation, depressive, anxiety, blood pressure regulation, gastrointestinal, eating and appetite disorders. Indeed, drugs known to interact with CB1 receptors are useful in the treatment or prevention of the aforementioned conditions.

In recent years a number of studies have suggested the existence of additional cannabinoid receptors. Calignano et al, postulated the existence of an SR144528-sensitive non-CB1/CB2 cannabinoid receptor (1998, Nature, 394:277-281). They presented evidence that although palmitoylethanolamide lacks significant affinity for CB1 or CB2 receptors, its ability to produce anti-hyperalgesia is readily attenuated by the CB antagonist/inverse agonist, SR144528. The presence of cannabinoid non-CB1/CB2 receptors in the mouse vas deferens has also been proposed. Evidence has also been obtained for the presence in vascular endothelium of an SR141716-sensitive non-CB1, non-CB2, non-vanilloid receptor that is unresponsive to established non-eicosanoid CB1/CB2 receptor agonists but can be activated by both eicosanoid cannabinoids, anandamide and methanandamide, as well as by certain classical cannabinoids (“abnormal cannabidiol” and its more potent analogue, 0-1602) that do not act through CB or vanilloid receptors. Interestingly, two effects of abnormal cannabidiol, hypotension and mesenteric vasodilation, were found to be antagonized by the non-psychotropic classical cannabinoid, cannabidiol. So too was anandamide-induced mesenteric vasodilation. Finally, the existence in brain of non-CB1, non-CB2, SR141716-insensitive G protein-coupled receptors for anandamide has recently been proposed to explain results obtained from experiments with CB knockout mice.

In view of the role of CB 1 and CB2 receptors in various diseases, there is a desire and need in the art to identify additional non-CB1 and -CB2 receptors that may also be implicated in disease and to use such receptors, alone or as part of a panel of other cannabinoid receptors, to identify and profile the effects of potential therapeutic compounds capable of treating one or other of the many diseases and disorders mediated by cannabinoid receptors.

G protein-coupled receptors (GPCRs) constitute a family of proteins sharing a common structural organization characterized by an extracellular N-terminal end, seven hydrophobic alpha helices putatively constituting transmembrane domains, and an intracellular C-terminal domain. GPCRs bind a wide variety of ligands that trigger intracellular signals through the activation of transducing G proteins (Caron et al., 1993, Rec. Prog. Horm. Res., 48:277-290; Freedman et al., 1996, Rec. Prog. Horm Res., 51:319-353. More than 300 GPCRs have been cloned, and it is generally assumed than well over 1,000 of such receptors exist. Roughly 50-60% of all clinically relevant drugs act by modulating the functions of various GPCRs (Gudermann et al., 1995, J. Mol. Med., 73:51-63).

The cloning and sequence of the orphan GPCR GPR55 is disclosed in Sawzdargo et al. (Molecular Brain Research. 64:193-198, 1999). The sequence is also disclosed in WO00/23588 and WO 01/86305. The cDNA sequence of wtGPR55 is disclosed in SEQ ID NO: 9. The encoded amino acid sequence is disclosed in SEQ ID NO:2.

### BRIEF DESCRIPTION OF THE DRAWINGS

FIG. 1 shows the phylogenic relationship of CB1 and CB2 to GPR55.

FIG. 2 shows the amino acid sequence of the human GPR55 variant, GPR55A (SEQ ID NO: 1), with a comparison to the sequence of GenBank accession number AF096786. The protein sequence for accession number AF096786 is distinct from GPR55A in that it contains the stretch of amino acids, SGPPGRSLGSA (SEQ ID NO:2), as shown in the figure. Also shown are the positions and changes of four different amino acid substitutions (# 4, 5, 10, and 12) that we have identified.

FIG. 3 shows the specific binding of cannabinoid radioligands to membranes prepared from GPR55 and GPR55A transfected cells.

FIG. 4 shows the concentration dependent displacement of [³H]-SR141716 by SR141716 using membranes expressing CB1, GPR55 and GPR55A.

FIGS. 5A-5D show GTPγS measurement of GPR55A activation in the presence of various concentrations of cannabinoid ligands.

FIG. 6 shows concentration response curves for CP55940 on GPR55a expressed in HEK293s membranes treated with either pertussis toxin, cholera toxin or untreated. The concentration dependent activity remains unaltered whilst the background activity of the system is lowered.

### DETAILED DESCRIPTION

We have discovered that the orphan G protein-coupled receptor GPR55, and variant sequences thereof, binds and is activated by endogenous, natural and synthetic cannabinoid ligands. We have therefore discovered that GPR55 (and variant sequences thereof) is a novel cannabinoid receptor and a biological and pharmaceutical target of cannabinoid ligands. GPR55, as a member of class A of the G-protein coupled receptor family, possesses the classical motifs associated with this family, including seven putative hydrophobic transmembrane helices and a (E/D)R(YFS) sequence at the cytoplasmic end of transmembrane helix 3. Phylogenically, GPR55 it is poorly related to other receptors, showing closest identity (27.2%) to another orphan receptor, GPR35. Surprisingly, GPR55 has low sequence identity with the cannabinoid receptors CB1 (13.5%) and CB2 (14.4%).

The present invention is based upon our discovery that GPR55 displays high affinity for endogenous, natural and synthetic cannabinoid ligands and for ligands including, but not limited to, palmitoylethanolamine (PEA), a potent anti-inflammatory and anti-hyperalgesic molecule, and virohdamine. We have discovered that PEA and virohdamine, as well as anandamine, noladin ether, 2-aracadonylglycerol and oleylethanolamine, bind with high affinity to GPR55 and can activate this receptor. Pharmacological properties of these ligands can, therefore, be mediated by the modulation of GPR55 activity.

The interaction between cannabinoid and other ligands and the GPR55 receptor can be harnessed in a variety of assays to identify compounds that modulate the binding of cannabinoid and other ligands to GPR55, to identify compounds that modulate cannabinoid and other ligand activation of GPR55, to identify compounds that are agonists, antagonists, allosteric modulators, or inverse agonists of GPR55, and to identify compounds that selectively modulate GPR55, rather than other cannabinoid receptors such as CB1 or CB2. Assays of the present invention can also be used to identify compounds having activity at any combination of CB1, CB1a, CB1b, CB2 and GPR55.

Modulation of GPR55 activity by endogenous, natural or synthetic agonists, antagonists or inverse agonists may be useful for the treatment (therapeutic or prophylactic) of a number of diseases where cannabinoid ligands play a key role or have a beneficial effect, in particular but not limited to tissues where GPR55A is expressed and where cannabinoids are implicated to have a significant disease modifying effect, such as the prefrontal cortex, substantia nigra and nucleus basalis of Meynert in CNS and cognition disorders, eg. schizophrenia, Alzheimers disease and dementia, or the caudate and putamen in Parkinson's disease and depression. Also, in the heart and the cardiovascular system, the cannabinoids can have a disease modifying effect particularly through GPR55A on vascular tone and atherosclerotic lesions and also, but not limited to, myocardial infarction, congestive heart failure, coronary heart disease, hypertension, angina, stroke, arrythmia, peripheral vascular disease, renal disease and other vascular sequelaes of diabetes. Also, in the GI-tract the cannabinoids can have modulating effects through this receptor particularly on gastro-intestinal motility related disorders such as IBS, IBD and GERD. Also cancers of GI-tract such as colon cancer, adenocarcinoma of the intestines and of the pancreas and liver in particular may be affected through cannabinoid modulation of this receptor. All forms of cancers of the CNS, in particular astrocytoma and glioblastoma and all forms of cancer of the lung, in particular adenocarcinomas, may be affected through cannabinoid modulation of this receptor. Compounds identified in using the assays of the present invention are furthermore useful for the treatment of obesity, e.g. by reduction of appetite and body weight, maintenance of weight reduction and prevention of rebound; psychiatric disorders such as psychotic disorders, e.g. schizophrenia and bipolar disorders, anxiety, anxio-depressive disorders, cognitive disorders, memory disorders, obsessive-compulsive disorders, anorexia, bulimia, attention disorders like ADHD, epilepsy, and related conditions; and, neurological disorders such as, multiple sclerosis, Raynaud's syndrome and Huntington's chorea. Such compounds are also potentially useful for the treatment of immune, cardiovascular, reproductive and endocrine disorders, septic shock and diseases related to the respiratory and gastrointestinal systems (e.g. diarrhea). Such compounds are also potentially useful as agents in treatment of extended abuse, addiction and/or relapse indications, e.g. treating drug (nicotine, ethanol, cocaine, opiates, etc) dependence and/or treating drug (nicotine, ethanol, cocaine, opiates, etc) withdrawal symptoms. Such compounds may also eliminate the increase in weight, which normally accompanies the cessation of smoking. Such diseases and disorders are herein referred to as cannabinoid receptor mediated diseases and disorders.

According to one aspect of the invention there is provided a method for determining whether or not a test compound may have potential in treating a cannabinoid receptor mediated disease or disorder comprising determining the modulatory effect that the test compound has on GPR55, wherein a compound with modulatory effect against GPR55 is one that has potential in treating a cannabinoid receptor mediated disease or disorder.

Particular examples of diseases and conditions that a modulatory compound against GPR55 may be used to treat include any of: hypertension, atherosclerosis and sequelas thereof, microvascular disease of Diabetes, Alzheimers disease, Parkinson disease, IBS, IBD, cancer of liver, cancer of pancreas and cancer of colon.

As used herein, the terms “modulate” or “modulates” in reference to binding include any measurable alteration to the binding interaction between a ligand and the GPR55 receptor, including, but not limited to, the amount or quantity of binding, binding affinity, and binding efficiency. For example, compounds identified using assays and methods of the present invention may increase or decrease the amount of binding of a ligand to the GPR55 receptor. Compounds identified using assays and methods of the present invention may enhance or inhibit the rate of binding of a ligand to the GPR55 receptor.

As used herein, the term “inhibit” in reference to binding of a ligand to the GPR55 receptor means any measurable decrease in binding.

As used herein, the term “decrease” in reference to cell stimulating activity or in reference to binding of a ligand to the GPR55 receptor means any measurable diminution of such cell stimulating activity or binding activity.

As used herein, the term “increase” in reference to cell stimulating activity or in reference to binding of a ligand to the GPR55 receptor means any measurable enhancement of such cell stimulating activity or binding activity.

As used herein, the terms “contact” or “contacting” refers to any method of combining components, such as combining compounds and/or a ligand in culture medium containing cells expressing the GPR55 receptor, or combining compounds and/or a ligand in solutions containing the GPR55 receptor, which may or may not be bound to a substrate.

As used herein, the phrase “functional fragment” in reference to the GPR55 receptor protein, refers to portions or fragments of the GPR55 receptor protein that are functionally active in the assays of the present invention, i.e., are capable of binding to and/or being activated by a ligand (for example, a cannabinoid ligand). Functional fragment also includes fusion proteins that contain portions of the GPR55 receptor.

As used herein, the term “variant” in reference to the GPR55 receptor protein includes proteins having amino acid modifications, mutations, deletions, or insertions and other protein modifications that retain functionality in the assays of the present invention. Variant GPR55 receptor proteins include, but are not limited to, GPR55A, which is described further herein below. In one embodiment the variant possesses no more than 20, preferably no more than 10, and still more preferably no more than 5 amino acid substitutions from the sequence depicted in SEQ ID NO: 1 or 2.

One skilled in the art can readily determine whether a protein or peptide is a functional fragment of the GPR55 receptor by examining its sequence and testing for binding and/or activation activity without undue experimentation. For example, binding to a ligand known to bind GPR55, such as those identified in the examples herein, or the ability to activate Gα₁₃-protein. Truncated versions of the GPR55 receptor and fusion proteins containing portions of the GPR55 receptor may be prepared and tested using routine methods and readily available starting material. Typically, a polypeptide with at least 80% identity, particularly at least 85%, at least 90%, at least 95%, at least 97% or at least 99% identity with the amino acid sequence of SEQ ID NO: 1, are considered as GPR55 variants. Variant polypeptides therefore include naturally occurring allelic variants as well as non-naturally occurring (or engineered) variants.

The sequence identity between two sequences can be determined by pair-wise computer alignment analysis, using programs such as, BestFit, Gap or FrameAlign. A commonly used alignment tool is BestFit. In practice, when searching for similar/identical sequences to the query search, from within a sequence database, it is generally necessary to perform an initial identification of similar sequences using suitable software such as Blast, Blast2, NCBI Blast2, WashU Blast2, FastA, Fasta3 and PILEUP, and a scoring matrix such as Blosum 62. Such software packages endeavour to closely approximate the “gold-standard” alignment algorithm of Smith-Waterman. Thus, the preferred software/search engine programme for use in assessing similarity, i.e. how two primary polypeptide sequences line up is Smith-Waterman. For the purpose of this invention the percent identity between two amino acid or nucleotide sequences is determined using the algorithm of E. Meyers and W. Miller (CABIOS, 4:11-17 (1989)) which has been incorporated into the ALIGN program (version 2.0), using a PAM120 weight residue table, a gap length penalty of 12 and a gap penalty of 4.

As used herein, the term “heterologous” in reference to the GPR55 receptor gene means any non-endogenous GPR55 receptor gene, for example, one that has been introduced or transfected into a cell, which includes GPR55 receptor genes from different species or organisms than the cell and recombinant GPR55 receptor genes from the same species or organism as the cell.

The present invention is also directed to an isolated nucleic acid encoding the polypeptide comprising the sequence depicted in SEQ ID NO: 1. The present invention also relates to the isolated polypeptide encoded by said nucleic acid.

The present invention provides methods of identifying compounds that modulate the binding interaction of the GPR55 receptor or any variant or functional fragment thereof, including GPR55A, with a cannabinoid, or other ligand capable of binding to the GPR55 receptor. The binding interaction can be monitored by direct measurement of binding or by measurement of a signal indicative of receptor activation or cell stimulating activity.

Examples of signals that can be measured in assays of the present invention and which serve as indicators of receptor activation or indicators of cell stimulating activity include, but are not limited to, intracellular phospholipase C (PLC) activity, phospholipase A (PLA) activity, adenylyl cyclase activity, cAMP levels, MAP kinase activity, GTPγS binding, and intracellular concentration of calcium in the cell, and opening and closing of ion channels.

Activation of the GPR55 receptor can be assayed by examination of a number of cellular signaling systems including, but not limited to, methods for measuring cAMP levels (alpha screen, reporter gene, scintillation proximity assays, etc.), methods for measuring intracellular calcium levels (FLIPR, use of promiscuous G-proteins such as G₁₆, G_{qi5}, G_{qs5}, etc.), methods for determining the activity of protein kinase A, protein kinase C, small G-protein activation, phospholipase C, small G-protein activation, and modulation of ion channel activity and function.

Many other methods of measuring receptor activation and cell stimulation are known to those skilled in the art and can be used in the assays of the present invention

According to a first aspect of the invention there is provided a method for identifying compounds that modulate binding of a cannabinoid or other ligand to the GPR55 receptor, said method comprising: providing cells expressing the GPR55 receptor or functional fragment or variant thereof; contacting said cells with the cannabinoid or other ligand, in the presence or absence of a compound; and measuring a signal indicative of receptor activation; where an alteration to said signal in the presence of a compound identifies said compound as a compound that modulates binding of the cannabinoid or other ligand to the GPR55 receptor.

According to a further aspect of the invention there is provided a method for identifying compounds that modulate the binding of a cannabinoid or other ligand to the GPR55 receptor, said method comprising: providing the GPR55 receptor or functional fragment or variant thereof; contacting the GPR55 receptor or functional fragment or variant thereof, with a cannabinoid or other ligand in the presence or absence of a compound; and measuring the amount of cannabinoid or other ligand that forms a complex with the GPR55 receptor or functional fragment or variant thereof; where an alteration to the amount of said complex formed in the presence of said compound identifies said compound as a compound that modulates binding of cannabinoid or other ligand to the GPR55 receptor.

In one embodiment, the GPR55 receptor/ligand complexes are isolated prior to measuring the amount of ligand in said complexes.

In other embodiments, the cannabinoid or other ligand is detectably labelled, for example, radiolabled, fluorescently labelled, or chemiluminescently labelled.

In another embodiment, the cannabinoid or other ligand is bound to an enzyme and measurement is carried out by enzyme-linked immunosorbent assay (ELISA).

In another embodiment, the GPR55 receptor, or functional fragment or variant thereof, is provided as cells expressing the GPR55 receptor or functional fragment or variant thereof, or is provided as membranes prepared from said cells.

According to a further aspect of the invention there is provided a method of screening for a GPR55 receptor agonist or antagonist comprising measuring a cell stimulating activity through a GPR55 receptor determined from the following steps a) and/or b): a) contacting a compound with a cell expressing a GPR55 receptor or functional fragment or variant thereof (test screen), and comparing the results to a control screen wherein the cell does not express the GPR55 receptor or functional fragment or variant thereof, wherein said compound having cell stimulating activity in the test screen but not the control screen indicates that the test compound is a GPR55 receptor agonist, b) contacting cannabinoid or other ligand and a test compound with a cell expressing a GPR55 receptor or functional fragment or variant thereof (test screen), and comparing the results to a control screen wherein the cell does not express the GPR55 receptor or functional fragment or variant thereof, where a decrease in cell stimulating activity by the cannabinoid or other ligand in the test screen but not the control screen indicates that the test compound is a GPR55 receptor antagonist.

According to a further aspect of the invention there is provided a method of screening for compounds that inhibit binding of cannabinoid or other ligand to the GPR55 receptor, comprising comparing the amount of cannabinoid or other ligand bound to the GPR55 receptor or functional fragment or variant thereof in steps a) and b): a) contacting cannabinoid or other ligand with the GPR55 receptor or functional fragment or variant thereof; b) contacting cannabinoid or other ligand and a test compound with the GPR55 receptor or functional fragment or variant thereof; where a decrease in cannabinoid or other ligand binding in step b) indicates that the test compound inhibits binding of the cannabinoid or other ligand to the GPR55 receptor.

According to a further aspect of the invention there is provided a method of identifying a compound that modulates binding of cannabinoid or other ligand to the GPR55 receptor, comprising contacting the GPR55 receptor or functional fragment or variant thereof with a cannabinoid or other ligand in the presence or absence of a test compound, and comparing the amount of binding between the cannabinoid or other ligand and the GPR55 receptor or functional fragment or variant thereof in the presence or absence of the test compound, where an alteration in the amount of binding between the cannabinoid or other ligand and the GPR55 receptor or functional fragment or variant thereof in the presence of the test compound indicates that the test compound modulates binding between the cannabinoid or other ligand and the GPR55 receptor.

According to a further aspect of the invention there is provided a method of identifying a compound that binds the GPR55 receptor, comprising incubating a cell expressing GPR55 receptor or functional fragment or variant thereof with a cannabinoid or other ligand in the presence or absence of a compound, and detecting displacement of cannabinoid or other ligand binding to the GPR55 receptor or functional fragment or variant thereof in the presence of the compound, where displacement of said binding is indicative of a compound that binds the GPR55 receptor.

According to a further aspect of the invention there is provided a method of determining if a test compound is an agonist, antagonist or inverse agonist of the GPR55 receptor comprising: a) incubating a cell expressing GPR55 or functional fragment or variant thereof with the test compound; b) measuring a signal indicative of receptor activation; and c) comparing the measurement in b) with a second measurement of a signal indicative of receptor activation obtained from incubations performed in the absence of the test compound, where the test compound is an agonist of GPR55 if the signal indicative of receptor activation is higher in the presence of the test compound than in its absence, and wherein the test compound is an antagonist of GPR55 if the signal indicative of receptor activation is lower in the presence of the test compound than in its absence.

Any ligand that binds and/or activates the GPR55 receptor or variant or functional fragment or thereof can be used with the assays of the present invention. Such ligands include, but are not limited to cannabinoid ligands (such as endocannabinoids (endogenous), natural, and synthetic). As used herein, the terms “cannabinoid ligand” and “GPR55 ligand” are used interchangeably to refer to such compounds that bind to and/or interact with and/or activate the GPR55 receptor or variant or functional fragment thereof. Any endocannabinoid can be used with the methods of the present invention, including, but not limited to, anadamide, 2-arcadonylglycerol, noladin ether, palmitoylethanolamine, and virodamine. Any plant-derived cannabinoid can be used with the methods of the present invention, including, but not limited to, Δ9-THC, cannabinol, and cannabidiol. Any plant-derived synthetic cannabinoid can be used with the methods of the present invention, including, but not limited to, CP55940, WIN55-212-2, HU210, AM251, AM281, AM630, JWH015, JWH133, ACEA, and ACPA. Radio-labelled cannabinoid receptor ligands that can be used with the methods of the present invention, include, but not limited to, [³H]-SR141716, [³H]-WIN55-212-2, [³H]-CP55940, [³H]-anandamide. Examples of suitable ligands for use in the screening methods of the invention include: anandamide, virohdamine, noladin ether, 2-aracodonylglycerol, palmityolethanolamine, oleylethanolamine and Δ9-tetrahydrocannbinol.

In some embodiments, cells expressing the GPR55 receptor are used in assays to identify compounds that modulate cannabinoid ligand/GPR55 binding. Cells expressing the GPR55 receptor can be incubated with a cannabinoid ligand and a test compound. Any cell type in which the GPR55 receptor is expressed, or can be engineered to be expressed, can be used. Any cell type in which receptor binding and/or receptor activation can be measured may be used in the assays of the invention. By way of non-limiting examples, the assay may utilize mammalian cells (including, but not limited to, human, hamster, mouse, rat, or monkey) or non-mammalian cells such as amphibian (e.g., frog) or fish cells. Cell lines that may be used in the assays of the invention include, but are not limited to, HEK-293s (human embryonic kidney), CHO (Chinese hamster ovary), LTk- (murine fibroblasts lacking cytosolic deoxythymidine kinase (dTK)), HeLa, BALB/c-3T3, Xenopus oocytes; melanophores (cells from fish and amphibians) may also be used. In some embodiments, HEK-293s cells expressing the G protein G_{α16} are used. Human cells are a preferred embodiment.

In some embodiments, a recombinant cell expressing a heterologous GPR55 receptor from a heterologous gene expression construct is used. Any species of GPR55 receptor may be used, including, but not limited to a mammalian GPR55 receptor, including human, rodent, murine, rat, guinea pig, mouse, hamster, rhesus, cynomologous monkey, and porcine. Human GPR55 is a preferred form The GPR55 receptor protein may be a fusion protein or may have variation in amino acid sequence, including deletions, insertions, mutations, and polymorphisms.

Another aspect of the invention relates to methods of determining if a test compound is an agonist, antagonist, allosteric modulator, or inverse agonist of GPR55 receptor binding based upon a functional assay. In some embodiments, assays are carried out by incubating a cell expressing GPR55 receptor or a functional fragment or variant thereof with a test compound and determining whether intracellular phospholipase C, adenyl cyclase activity, cAMP levels, MAP kinase activity, GTPγS binding or intracellular calcium concentrations are modulated. Results can be compared with controls wherein incubations are performed in a similar manner but in the absence of the test compound. Functional assays of this type can be performed in conjunction with binding assays, including those described herein. In some embodiments, the cell used in functional assays is a recombinant cell that has been transformed with a heterologous GPR55 receptor gene or construct encoding GPR55 or functional fragment or variant thereof.

Test compounds that act as agonists are indicated by an increase in phospholipase C, a decrease or increase in adenylyl cyclase activity, a decrease or increase in cAMP levels, an increase in intracellular calcium levels, or an increase in the level of GTPγS binding.

Inverse agonists can reduce phospholipase C activity or intracellular calcium levels, particularly if assays are performed in the presence of a fixed amount of ligand. Antagonists block binding of ligand to the receptor but do not produce the opposite response in terms of phospholipase C activity, GTPγS binding levels, or intracellular calcium, that is the hallmark of an inverse agonist.

In some embodiments of the invention the GPR55 receptor is recombinantly expressed in cells from a heterogenous or heterologous gene. Nucleic acid sequences encoding the human GPR55 receptor can be cloned as described by Sawzdargo et al., 1999, Mol. Brain. Res., 64 193-198 (see also, GenBank accession number AF096786, WO 01/86305, and WO 00/23588, all incorporated herein by reference).

The GPR55 receptor coding sequence can be incorporated into an expression vector with a promoter and other regulatory elements that will be active and appropriate for expression in the particular cell type used (see, Sambrook et al., eds., Molecular Cloning: A Laboratory Manual (2^{nd} ed.), Cold Spring Harbor Laboratory Press, Cold Spring Harbor, N.Y. (1989)). In some embodiments, mammalian cells are used. Examples of promoters that may be used for expression in mammalian cells, include, but are not limited to, the mouse metallothionein I gene promoter (Hamer et al., 1982, J. Mol. Appl. Gen., 1:273-288), the immediate-early and TK promoter of herpes virus (Yao et al., 1995, J. Virol., 69:6249-6258, McKnight, 1982, Cell, 31:355-365); the SV40 virus early promoter (Benoist et al., 1981, Nature, 290:304-310), and the CMV promoter (Boshart et al., 1985, Cell, 41:521-530). Vectors may also include enhancers and other regulatory elements.

Expression vectors can be introduced into cells by methods well known to the art, including, but not limited to, calcium phosphate precipitation, microinjection, electroporation, liposomal transfer, viral transfer, or particle-mediated gene transfer.

In some embodiments the GPR55 receptor is used to screen for compounds that mimic the action of cannabinoid or other GPR55 receptor ligands (agonists).

In some embodiments the GPR55 receptor is used to screen for compounds that antagonize the action of cannabinoid or other GPR55 receptor ligands (antagonists).

In some embodiments of any aspect of the invention, the human GPR55 receptor is used.

In some embodiments of any aspect of the invention, the GPR55A receptor is used.

Cells can be selected and assayed or examined for the expression of the GPR55 receptor according to standard procedures and techniques known to the art, including, but not limited to Northern blotting analysis.

In some embodiments, cells expressing the GPR55 receptor are used in conjunction with a cannabinoid ligand in screening assays designed to identify compounds that modulate cannabinoid ligand/GPR55 binding. Cells expressing the GPR55 receptor can be incubated with a cannabinoid ligand and a test compound. The extent to which the binding of the cannabinoid ligand is displaced by the test compound is then determined. Radioligand assays or enzyme-linked immunosorbent assays may be performed in which either the cannabinoid ligand or the test compound is detectably labelled. In certain embodiments, the cannabinoid or other ligand is radiolabelled, fluorescent labelled or chemiluminescent labelled.

In some embodiments, a cannabinoid or other ligand and cells expressing the GPR55 receptor are used in assays to determine whether test compounds have any modulatory effect on the binding between a cannabinoid or other ligand and the GPR55 receptor. A wide variety of different types of assays can be performed using standard methods known to those of skill in the art. For example, in radioligand binding assays, cells expressing the GPR55 receptor are incubated with a cannabinoid or other ligand and with a compound being tested for binding activity. In some embodiments, the source of the GPR55 receptor is recombinantly transformed HEK-293s cells. In some embodiments, other cells types are utilized, including cell types that do not express other proteins that bind the cannabinoid or other ligand being utilized. Such cell types can easily be determined by performing binding assays on cells transformed with GPR55 and comparing the results obtained with those obtained using their non-transformed counterparts.

In some embodiments of the invention, functional assays, such as mobilization of intracellular calcium, are carried out using a FLIPR (Fluorescent Imaging Plate Reader) detection system

In some embodiments of the invention, the cannabinoid or other ligand is iodinated and used as a tracer in radioligand binding assays on whole cells or membranes. Other assays that can be used include, but are not limited to, the GTPγS assay, adenylyl cyclase assays, assays measuring inositol phosphates, and reporter gene assays (e.g., those utilizing luciferase, aqueorin, alkaline phosphatase, etc.).

Assays may be performed using either intact cells or membranes prepared from the cells (see e.g., Wang et al., Proc. Natl. Acad. Sci. U.S.A. 90:10230-10234 (1993)). In some embodiments, membranes or whole cells are incubated with a cannabinoid or other ligand and with a preparation of the compound being tested. After binding is complete, the receptor is separated from the solution containing the ligand and test compound, e.g., by filtration, and the amount of binding that has occurred is determined. In some embodiments, the ligand used is detectably labelled with a radioisotope such as, for example, ¹²⁵I. Other types of labels can also be used, including, but not limited to, the following fluorescent labeling compounds: fluorescein, isothiocynate, rhodamine, phycoerythrin, phycocyanin, allophycocyanin o-phthaldehyde and fluorescamine. Chemiluminescent compounds can also be used with the assays of the invention, including, but not limited to, luminol, isoluminol, theromatic of acridinium ester, imidazole, acridinium salt, and oxalate ester.

In some embodiments of the invention, assays are performed in a cell-free environment, such as, for example, where only the binding interaction between a cannabinoid or other ligand and the GPR55 receptor is being examined. In such cell-free or in vitro binding assays the cannabinoid or other ligand and/or the GPR55 receptor may be bound to a support.

In some embodiments of the invention, assays are carried out wherein the compound is tested at different concentrations and the signal measured at these different concentrations permits the binding affinity of the compounds to be determined.

Non-specific binding may be determined by carrying out the binding reaction in the presence of a large excess of unlabelled ligand. For example, labelled cannabinoid or other ligand may be incubated with GPR55 receptor and test compound in the presence of a thousand-fold excess of unlabelled cannabinoid ligand. Nonspecific binding can be subtracted from total binding, i.e., binding in the absence of unlabelled ligand, to arrive at the specific binding for each sample tested Other steps such as washing, stirring, shaking, filtering and the like can be included in the assays as necessary. Typically, wash steps are included after the separation of membrane-bound ligand from ligand remaining in solution and prior to quantitation of the amount of ligand bound, e.g., by counting radioactive isotope. The specific binding obtained in the presence of test compound is compared with that obtained in the presence of labelled ligand alone to determine the extent to which the test compound has displaced receptor binding.

In performing binding assays, artifacts may falsely make it appear that a test compound is interacting with receptor when, in fact, binding is being inhibited by some other mechanism. Such artefact-generated false signals can be dealt with in a number of ways known to those of skill in the art. For example, the compound being tested can be placed in a buffer which does not itself substantially inhibit the binding of cannabinoid or other ligand to the GPR55 receptor, and compounds can be tested at several different concentrations. Preparations of test compounds can be examined for proteolytic activity and antiproteases can be included in assays. Additionally, compounds that are identified as displacing the binding of cannabinoid or other ligand to the GPR55 receptor can be re-examined in a concentration range sufficient to perform a Scatchard analysis on the results. This type of analysis is known to those of ordinary skill in the art and can be used for determining the affinity of a test compound for a receptor (see e.g., Ausubel et al., eds., Current Protocols in Molecular Biology, 11.2.1-11.2.19, John Wiley & Sons, New York, N.Y. (1993); Work et al., eds., Laboratory Techniques in Biochemistry and Molecular Biology, NY (1978)). Computer programs can be used to assist in the analysis of results (e.g., Munson, 1983, Methods Enzymol., 92:543-577).

Depending upon their effect on the activity of the receptor, agents that inhibit the binding of the cannabinoid or other ligand to the GPR55 receptor may be either agonists or antagonists. Activation of the GPR55 receptor may be monitored using a number of different methods. For example, phospholipase C assays may be performed by growing cells in wells of a microtiter plate and then incubating the wells in the presence or absence of test compound total inositol phosphates (IP) may then be extracted in resin columns, and resuspended in assay buffer. Assay of IP thus recovered can be carried out using any method for determining IP concentration. Typically, phospholipase C assays are performed separately from binding assays, but it is also possible to perform binding and phospholipase C assays on a single preparation of cells.

Receptor activation can also be determined based upon a measurement of intracellular calcium concentration. Many types of assays for determining intracellular calcium concentrations are well known to the art and can be employed in the methods of the invention. For example, transformed HEK-293s can be grown to confluence on glass cover slides. After rinsing, the cells can be incubated in the presence of an agent such as Fluo-3, Fluo-4, or FURA-2 AM (Molecular Probes, Eugene, Oreg.). After rinsing and further incubation, calcium displacement can be measured using a photometer.

Assays that measure the intrinsic activity of the receptor, such as those based upon inositol phosphate measurement, can be used to determine the activity of inverse agonists. Unlike antagonists that block the activity of agonists but produce no activity on their own, inverse agonists produce a biological response diametrically opposed to the response produced by an agonist. For example, if an agonist promoted an increase in intracellular calcium, an inverse agonist would decrease intracellular calcium levels.

The radioligand and cell activation assays described herein provide examples of the types of assays that can be used for determining whether a particular test compound alters the binding of the cannabinoid or other ligand to the GPR55 receptor and acts as an agonist or antagonist. There are many variations on these assays that are compatible with the present invention. Such assays can involve the use of labelled antibodies as a means for detecting the cannabinoid or other ligand that has bound to the GPR55 receptor or may take the form of the fluorescent imaging plate reader assays.

Another aspect of the present invention is directed to screening methods using GPR55 or any variant or functional fragment thereof, including GPR55A, in a matrix fashion with CB1 CB1a, CB1b and CB2, in order to identify compounds having discrete pharmacological profiles at these receptors. In some embodiments, the present invention provides assays to identify compounds that act selectively as agonists/antagonists/inverse agonists/allosteric modulators at only one of the cannabinoid receptors. In further embodiments, the present invention provides assays to identify compounds having activity at any two of these receptors. In still further embodiments, the present invention provides assays to identify compounds that act as agonists at one of the receptors but as antagonists at one of the others. Given the diverse roles of cannabinoids in biological processes, the assays of the present invention are useful for the identification and development of compounds with selective effect profiles. Compounds that selectively modulate a particular cannabinoid receptor may be particularly useful in treating distinct diseases mediated by the particular receptor without adversely affecting the normal physiology mediated by other receptors. In this way, compounds may be tailored to particular diseases and side-effects kept to a minimum.

According to another aspect of the invention there is provided a screening system wherein the modulatory ability of a test compound is determined by screening the compound against a panel of cannabinoid receptors, said panel comprising GPR55 or any variant or functional fragment thereof, and at least one other cannabinoid receptor family member. Any of the screening methods disclosed herein could be used in this aspect of the invention.

In a particular embodiment, the “at least one other cannabinoid receptor family member” is selected from the group consisting of CB1, CB1a, CB1b and CB2.

According to a further aspect of the invention there is provided a method for determining the selectivity of a test compound against a cannabinoid receptor family member comprising determining the ability of the test compound to modulate each of a panel of cannabinoid receptors, said cannabinoid receptor panel comprising GPR55 or any variant or functional fragment thereof, and at least one other cannabinoid receptor selected form CB1, CB1a, CB1b and CB2.

A profile of the effects of the test compound against each receptor can then be generated. Such profile may in its simplest form be a table of data depicting the modulatory effect of a test compound against each receptor.

According to a further aspect of the invention there is provided the use of GPR55 receptor or any variant or functional fragment thereof, and at least one other receptor selected from the group consisting of: CB1, CB1a, CB1b and CB2, in a panel screen testing the modulatory ability of a test compound against each of the receptors in the panel.

According to a further aspect of the invention there is provided the use of a compound capable of selectively modulating GPR55 receptor in the treatment of a cannabinoid receptor mediated disease or condition.

According to a further aspect of the invention there is provided a method of treating a disease or condition selected from: hypertension, atherosclerosis and sequelas thereof, renal disease and microvascular disease of diabetes, Alzheimers, Parkinson disease, IBS, GERD and IBD, liver cancer, pancreatic cancer, colon cancer, astrocytoma and glioblastoma, by administering to a patient in need thereof an effective amount of a compound capable of selectively modulating GPR55 (a GPR55 selective compound). By selective we mean one exhibiting at least 10-fold more activity against GPR55 than either of CB1 or CB2.

According to a further aspect of the invention there is provided a method of treating a disease or condition selected from: hypertension, atherosclerosis and sequelas thereof, microvascular disease of Diabetes, Alzheimers disease, Parkinson disease, IBS, IBD, cancer of liver, cancer of pancreas and cancer of colon, by administering to a patient in need thereof an effective amount of a compound capable of selectively modulating GPR55.

Given the diverse roles of cannabinoids in biological processes, the assays of the present invention are useful for the identification and development of compounds with selective effect profiles.

The modulators identified according to the assays of the present invention are predicted to be of potential use in the treatment of certain of the cannabinoid receptor mediated diseases or disorders, such as those identified above.

The invention is further illustrated by way of the following examples, which are intended to elaborate several embodiments of the invention. These examples are not intended to, nor are they to be construed to, limit the scope of the invention. It will be clear that the invention may be practiced otherwise than as particularly described herein. Numerous modifications and variations of the present invention are possible in view of the teachings herein and, therefore, are within the scope of the invention.

### EXAMPLES

### Example 1

### Cloning of GPR55A

In order to test whether GPR55 has any cannabinoid or cannabinoid like activity we carried out PCR and amplified and sequenced isolated cDNAs.

GPR55-specific primers were designed based on the human GPR55 sequence (EMBL-AF096786) and were used for PCR amplification of the entire GPR55 open reading frame. PCR was carried out using human genomic DNA (Clontech (Palo Alto, Calif.) cat#6550-1, lot#45641) as the template and GPR55F and GPR55R primers (shown below). The product generated in the PCR reaction was subsequently used as a template for the GPR55flagF and GPR55flagR primers in a second round of PCR to introduce a Flag epitope as well as NheI and BamHI sites. DNA fragments including the Flag epitope were then inserted into the corresponding restriction sites of the pIRESneo2 vector (Clontech, Palo Alto, Calif.).

Primers

| (SEQ ID NO:3) | |
|---|---|
| GPR55F | 5′-ATG AGT CAG CAA AAC ACC AGT GGG GAC-3′ |
| (SEQ ID NO:4) | |
| GPR55R | 5′-TTA GCC CCG GGA GAT CGT GGT GTC-3′ |
| (SEQ ID NO:5) | |
| | NheI Kozak Start Flag |
| GPR55flagF | 5′-CAA GCT AGC TAT GCC ACC ATG GAC TAC AAG GAC |
| | GAC GAT GAC AAG AGT CAG CAA AAC ACC AGT |
| | GGG GAC-3′ |
| (SEQ ID NO:6) | |
| | BamHI Stop |
| GPR55flagR | 5′-CCT GGA TCC AAA TTA GCC CCG GGA GAT CGT |
| | GGT GTC-3′ |

This led to the identification of an insertion and deletion within the published nucleotide sequence (Sawzdargo et al. 1999, Mol Brain Res., 64 193-198; GenBank accession number AF096786) that resulted in a change of 11 amino acids in the putative region of intracellular loop 2 and TM4.

The effect of insertion and deletion on GPR55 protein sequence is as follows:

GenBank AF096786: . . . S G P P G R S L G S A . . . GPR55(SEQ ID NO:8)

Clone we obtained: . . . L R S P R K I F G I C . . . GPR55A (SEQ ID NO:7)

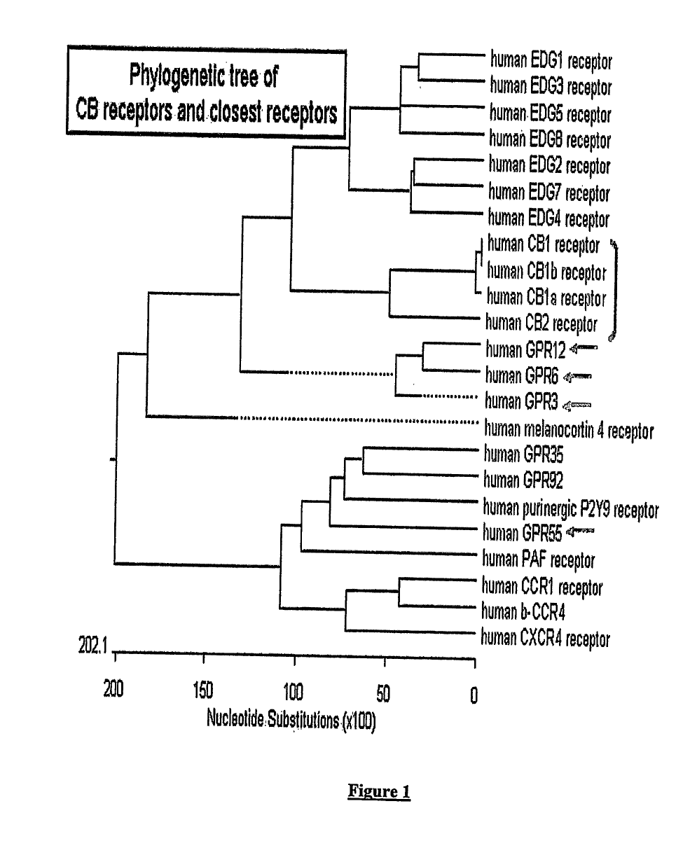

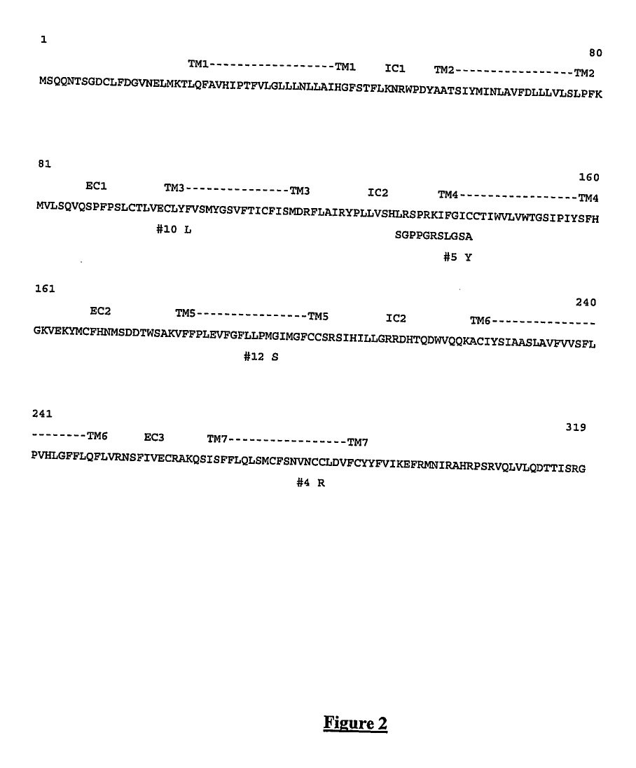

The alignment of this change can be seen in FIG. 2, which shows the complete protein sequence for GPR55A. This change, which we have named GPR55A, corresponds with the translation of the human genome sequence, and has also been identified in WO 00/23588. We have, however, also identified four other single nucleotide substitutions in our clones that result in single amino acid sequence substitution differences between GRP55A protein and the AF096786 protein sequence. These are also shown in FIG. 1 and are represented by # designations (#4, 5, 10, and 12).

Expression

Expression of GPR55A is achieved by transient transfection of appropriate plasmid DNAs using HEK293s cells or another appropriate cell line and Lipofectamine Plus or alternative transfection reagent, according to the manufacture's recommendations. Cells are harvested and membranes prepared as previously described (Milligan, G. (1999) Signal Transduction: A Practical Approach, pages 65-67, Oxford University Press).

### Example 2

### GRP55A has the Ability to Bind Cannabinoid Ligands when Expressed in HEK293s Cells

To test whether GPR55 and/or GPR55Δ encoded a receptor that had the potential to perform as a cannabinoid receptor we transiently transfected cDNAs encoding FLAG-tagged forms of the receptors into HEK293S cells using lipofectamine. The cells were harvested 48 hours later and membranes prepared.

Membrane Preparations

Membrane preparations were prepared by re-suspending receptor expressing cells in ice cold TE buffer (10 mM Tris-HCL 0.1 mM EDTA pH7.5) and leaving on ice for 5 minutes before pelleting the insoluble material by centrifugation at 1000 g. This process was repeated twice before re-suspending the pellet in an appropriate volume of TE and storing at −80° C.

A FLAG-tagged CB1 receptor-expressing construct was included as a positive control for the experiment, whilst empty vector was included as a negative control

In addition, transfections were performed for the purpose of immunohistochemistry to demonstrate that receptors were expressed at the cell surface (data not shown).

Radioligand Binding

Test compounds were added followed by 1.6 nM radioligand (3H—SR-141716A, ³H-CP55940 or ³H-WIN-55, 212-2) to an assay mixture containing membranes (10 μg protein) in 5.0 mM Tris, 5.0 mM MgCl₂×6H₂O (pH 7.4) supplemented with 0.05% BSA. The reaction was allowed to proceed at 30° C. for 90 min and then terminated by rapid filtration using GF/B filters (pre-soaked in 0.05% PEI) and washed with 50 mM Tris (pH 7.4), MgCl₂×6H₂O. Filters were then covered with scintillant and counted for the amount of radioligand retained. Non-specific binding was determined in the presence of 200 nM SR-141716A, CP55940 or WIN-55, 212-2.

Results

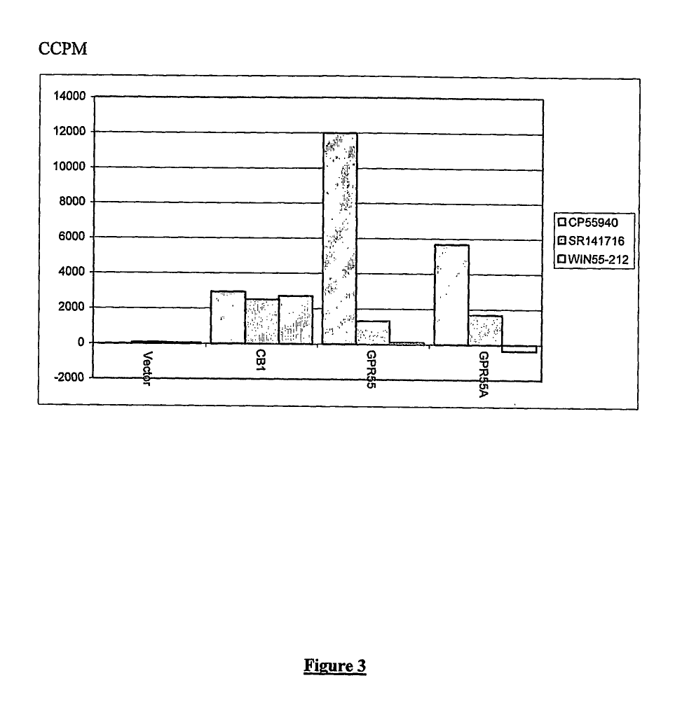

We chose three cannabinoid radioligands ([³H]-CP55940—non selective cannabinoid agonist; [³H]-WIN55-212-2-non-selective cannabinoid agonist; [³H]-SR141716, CB1 selective inverse agonist) as tools to test for specific binding in the GPR55 transfections using excess unlabelled of the same ligand to determine the level of non-specific binding. The results are shown in FIG. 3.

No specific binding was obtained for un-transfected cells (not shown) or vector transfected negative control cells. However, specific binding was obtained as expected for the positive control CB1 transfected cells with each of the radioligands. Membranes prepared from cells transfected with either GPR55 or GPR55A displayed significant binding for [³H]-CP55940 and [³H]-SR141716, but little or no binding of [³H]-WIN55-212, suggesting that GPR55 and GPR55A has some cannabinoid like ligand binding properties.

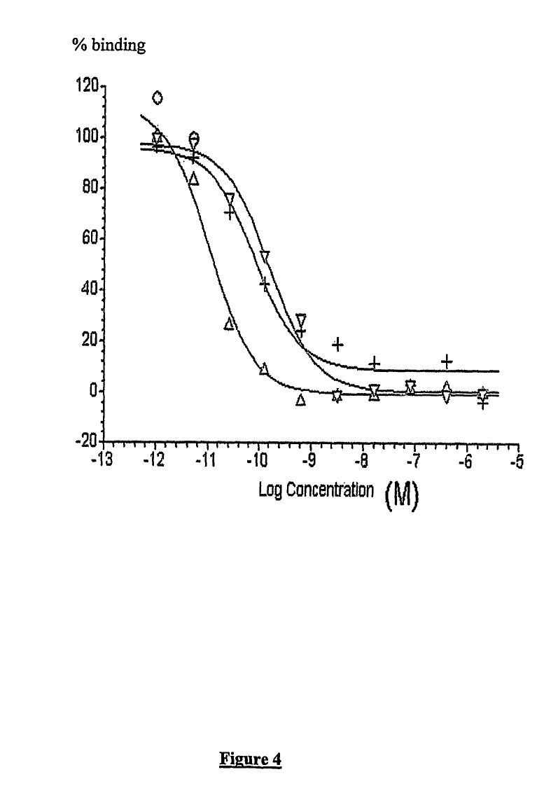

In order to further evaluate this binding, we generated competition-binding curves for [³H]-SR141716 being competed by the unlabelled version of itself against GPR55, GPR55A, CB1 and vector control membranes. The results are shown in FIG. 4. These data demonstrate an IC₅₀ of 0.3 nM for SR141716 at CB1, as expected, and no specific binding to membranes derived from vector-transfected cells was observed. GPR55 and GPR55A both displayed nanomolar affinity displacement curves.

### Example 3

### GPR55 and GRP55A have Functional Cannabinoid Activities

To investigate whether GPR55 or GPR55A possessed any cannabinoid-like functional activity, receptor-expressing cell membranes were tested in a GTPγS assay. Membranes with or without 20 μM ligand were assayed for their stimulatory or inhibitory activity on GTPγS binding. As a negative control, cell membranes were prepared from empty vector-transfected cells, and as a positive control, cell membranes were prepared from CB1 transfected cells.

GTPγS Binding Assay

Receptor activity was determined using a GTPγS binding assay as follows: 10 μg of membranes diluted in 200 μl of 100 mM NaCl, 5 mM MgCl₂, 1 mM EDTA, 50 mM HEPES (pH 7.4), 1 mM DTT, 0.1% BSA and 100 μM GDP. To this was added the required concentration of test compound and 0.11Ci ³⁵S-GTPγS in the presence or absence of an EC₈₀ concentration of agonist (HU210). The reaction was allowed to proceed at 30° C. for 45 min. Samples were then transferred on to GF/B filters using a cell harvester and washed with wash buffer (50 mM Tris (pH 7.4), 5 mM MgCl2, 50 mM NaCl). Filters were then covered with scintilant and counted for the amount of ³⁵S-GTPγS retained by the filter. To determine the level of non-specific binding control reactions were performed in the presence of 10 μM GTPγS.

Functional activity of ligands either in the presence or absence of agonists was determined as follows: Non-specific binding was subtracted from all values determined. The effect of compounds at various concentrations was plotted according to the equation
y=A+((B−A)/1+((C/x)?ˆD)))
and EC₅₀ or IC₅₀ estimated.
Results

No significant activity was detected for any compounds against membranes prepared from vector control transfections. The profile of compounds against CB1 was as expected for those ligands at CB1 receptors. Compounds acting at either GPR55 (data not shown) or GPR55A (see Table 1) displayed various effects upon GTPγS binding.

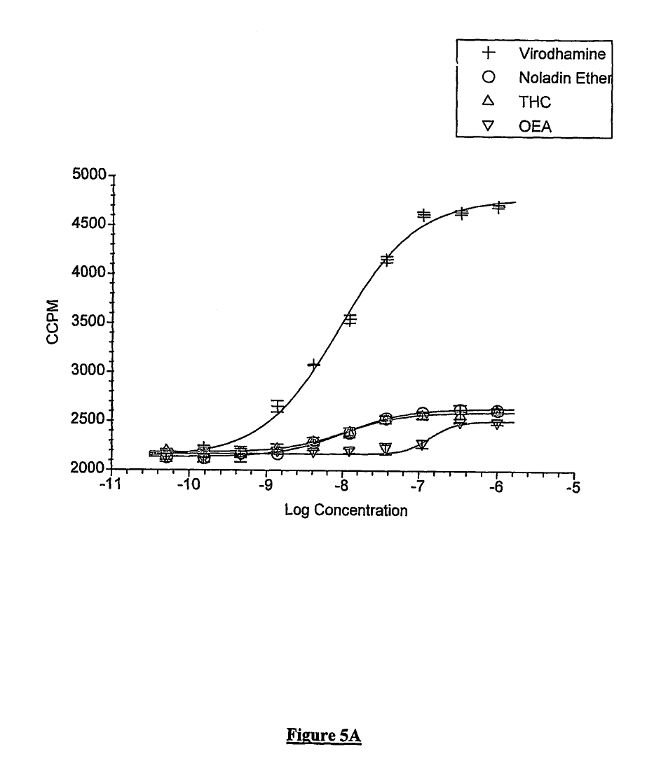

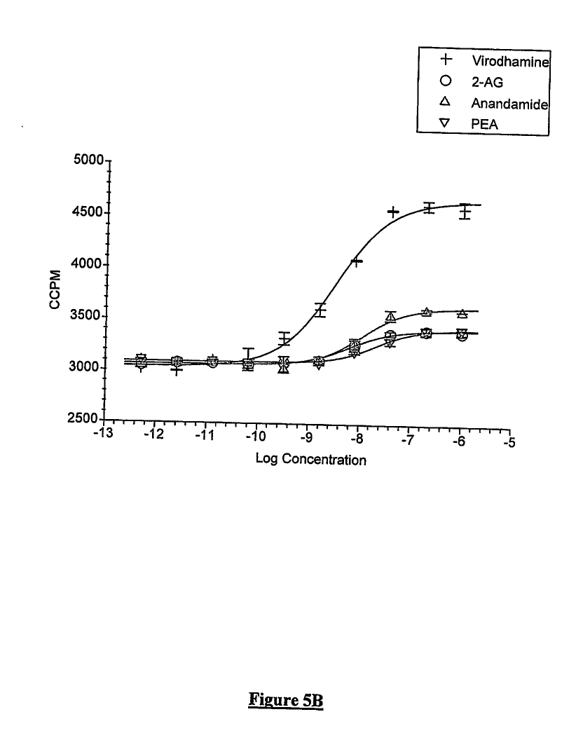

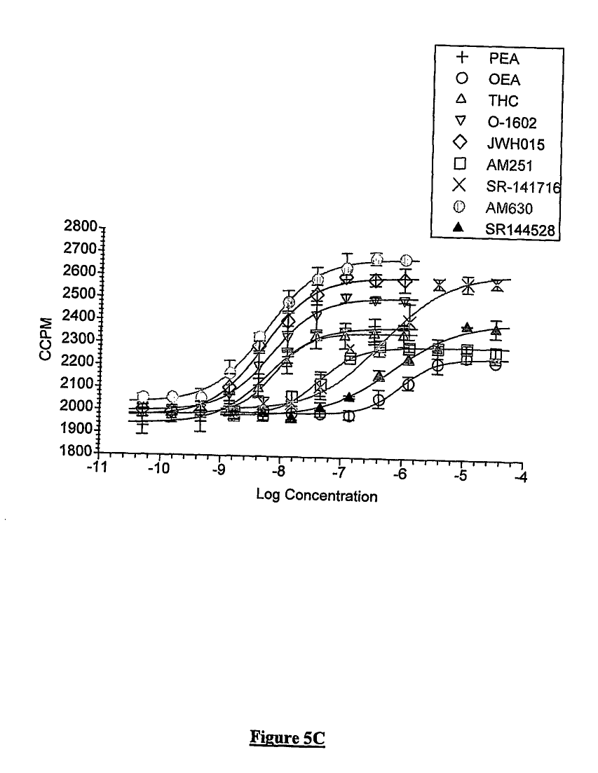

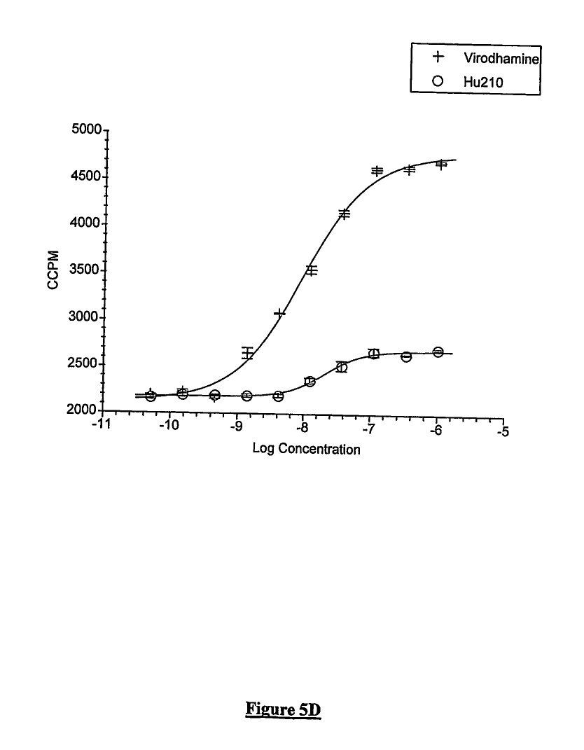

To further define the agonist activities of cannabinoid ligands EC₅₀ determinations were made. As before, none of the ligands displayed activity on control vector-transfected cells. Refer to FIGS. 5A-5D for the results. CB1-transfected cells generated EC₅₀ values comparable to those expected for each ligand (data not shown). GPR55A displayed concentration-dependent activation in response to the natural ligands anandamide, Noladin ether, 2-aracodonylglycerol, palmityolethanolamine and Δ9-tetrahydrocannbinol. The data for these and other compounds are summarized in Table 1. Table 1. Activity of ligands at GPR55A (Antagonist data were generated in the presence of an EC₈₀ concentration of HU210.)

| | EC₅₀ | |
|---|---|---|
| COMPOUND | (nM) (except*) | n |
| Anandamide | 18.4 ± 4.1 | 6 |
| Noladin Ether | 11.5 ± 0.5 | 2 |
| Palmitoylethanolamide | 3.2 ± 1.3 | 3 |
| Virodhamine | 10.4 ± 1.4 | 7 |
| 2-AG | 3.5 ± 2.4 | 2 |
| Oleylethanolamide | 424 ± 203 | 3 |
| Δ9-THC | 8.9 ± 1.1 | 3 |
| CP55940 | 7.1 ± 1.3 | 5 |
| Win55 | NA | 3 |
| Hu210 | 33 ± 12 | 2 |
| JWH-015 | 4.75 ± 0.25 | 2 |
| JWH-133 | NA | 2 |
| Cannabidiol | 354 ± 63* | 4 |
| Cannabinol | NA | 3 |
| Abnormal cannabidiol | 2780 ± 98 | 2 |

NA = not active
*IC₅₀ measurement

### Example 4

### Assessing Whether or not GPR55A Couples to Gᵢ or G₈ G Proteins

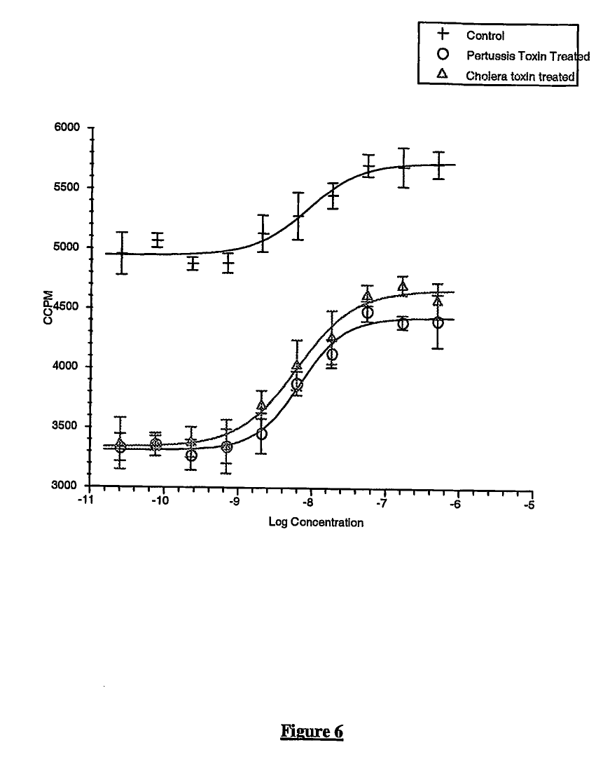

The use of pertussis and cholera toxins permits the selective inhibition of Gi and Gs G-protein activation. HEK293s cells transiently transfected with GPR55a were treated with either toxin and the membranes generated tested for their ability to generate a GTPγS signal FIG. 6 shows concentration response curves for CP55940 on GPR55a expressed in HEK293s membranes treated with either pertussis toxin, cholera toxin or untreated. The concentration dependent activity remains unaltered whilst the background activity of the system is lowered. Neither toxin was able to affect the GTPγS assay, demonstrating that GPR55a is coupled not to Gi or Gs in this system and therefore is probably coupled to another G-protein.

The foregoing examples are meant to illustrate the invention and are not to be construed to limit the invention in any way. Those skilled in the art will recognize modifications that are within the spirit and scope of the invention.

1. A method for identifying compounds that modulate binding of a cannabinoid or other ligand to the GPR55 receptor, said method comprising:
providing cells expressing the GPR55 receptor or functional fragment or variant thereof;
contacting said cells with the cannabinoid or other ligand, in the presence or absence of a compound; and,
measuring a signal indicative of receptor activation;
where an alteration to said signal in the presence of a compound identifies said compound as a compound that modulates binding of the cannabinoid or other ligand to the GPR55 receptor.

2. The method of claim 1, wherein the GPR55 receptor is expressed from a heterologous GPR55 receptor gene.

3. The method of claim 1, wherein the GPR55 receptor is mammalian.

4. The method of claim 3, wherein the GPR55 receptor is human.

5. The method of claim 1, wherein the GPR55 receptor is GPR55A.

6. The method of claim 1, wherein said cells are mammalian cells.

7. The method of claim 6, wherein the cells are human cells.

8. The method of claim 1, wherein said measuring is performed using a FLIPR assay.

9. The method of claim 1, wherein the signal indicative of receptor activation is selected from intracellular phospholipase C activity, phospholipase A activity, adenylyl cyclase activity, cAMP level, MAP kinase activity, GTPγS binding, and intracellular concentration of calcium.

10. A method for identifying compounds that modulate the binding of a cannabinoid or other ligand to the GPR55 receptor, said method comprising:
providing the GPR55 receptor or functional fragment or variant thereof;
contacting the GPR55 receptor or functional fragment or variant thereof, with a cannabinoid or other ligand in the presence or absence of a compound; and
measuring the amount of cannabinoid or other ligand that forms a complex with the GPR55 receptor or functional fragment or variant thereof;
where an alteration to the amount of said complex formed in the presence of said compound identifies said compound as a compound that modulates binding of cannabinoid or other ligand to the GPR55 receptor.

11. The method of claim 10, wherein GPR55 receptor/ligand complexes are isolated prior to measuring the amount of ligand in said complexes.

12. The method of claim 11, wherein the cannabinoid or other ligand is detectably labelled.

13. The method of claim 12, wherein the cannabinoid or other ligand is radiolabelled, fluorescently labelled, or chemiluminescently labelled.

14. The method of claim 13, wherein the cannabinoid or other ligand is radiolabelled.

15. The method of claim 10, wherein the cannabinoid or other ligand is bound to an enzyme, and measuring is carried out by enzyme-linked immunosorbent assay (ELISA).

16. The method of claim 10, wherein the GPR55 receptor, or functional fragment or variant thereof, is human.

17. The method of claim 10, wherein the GPR55 receptor is GPR55A.

18. The method of claim 10, wherein the GPR55 receptor, or functional fragment or variant thereof, is provided as cells expressing the GPR55 receptor or functional fragment or variant thereof, or is provided as membranes prepared from said cells.

19. The method of claim 18, wherein the GPR55 receptor, or functional fragment or variant thereof, is expressed from a heterologous GPR55 receptor gene.

20. The method of claim 18, wherein said cells are mammalian cells.

21. The method of claim 20, wherein the cells are human cells.

22. A method of screening for a GPR55 receptor agonist or antagonist comprising measuring a cell stimulating activity through a GPR55 receptor determined from the following steps a) and/or b):
a) contacting a compound with a cell expressing a GPR55 receptor or functional fragment or variant thereof (test screen), and comparing the results to a control screen wherein the cell does not express the GPR55 receptor or functional fragment or variant thereof, wherein said compound having cell stimulating activity in the test screen but not the control screen indicates that the test compound is a GPR55 receptor agonist,
b) contacting cannabinoid or other ligand and a test compound with a cell expressing a GPR55 receptor or functional fragment or variant thereof (test screen), and comparing the results to a control screen wherein the cell does not express the GPR55 receptor or functional fragment or variant thereof, where a decrease in cell stimulating activity by the cannabinoid or other ligand in the test screen but not the control screen indicates that the test compound is a GPR55 receptor antagonist.

23. The method of claim 22, wherein the signal indicative of receptor activation is selected from intracellular phospholipase C activity, phospholipase A activity, adenylyl cyclase activity, cAMP level, MAP kinase activity, GTPγS binding, and intracellular concentration of calcium.

24. A method of screening for compounds that modulate binding of a cannabinoid or other ligand to the GPR55 receptor, comprising comparing the amount of cannabinoid or other ligand bound to the GPR55 receptor or functional fragment or variant thereof in steps a) and b):
a) contacting a cannabinoid or other ligand with the GPR55 receptor or functional fragment or variant thereof;
b) contacting cannabinoid or other ligand and a test compound with the GPR55 receptor or functional fragment or variant thereof;
where an alteration in the amount of cannabinoid or other ligand bound to the GPR55 receptor or functional fragment or variant thereof in step b) indicates that the test compound modulates binding of the cannabinoid or other ligand to the GPR55 receptor.

25. A method of screening for compounds that inhibit binding of cannabinoid or other ligand to the GPR55 receptor, comprising comparing the amount of cannabinoid or other ligand bound to the GPR55 receptor or functional fragment or variant thereof in steps a) and b):
a) contacting cannabinoid or other ligand with the GPR55 receptor or functional fragment or variant thereof;
b) contacting cannabinoid or other ligand and a test compound with the GPR55 receptor or functional fragment or variant thereof;
where a decrease in cannabinoid or other ligand binding in step b) indicates that the test compound inhibits binding of the cannabinoid or other ligand to the GPR55 receptor.

26. A method of identifying a compound that modulates binding of cannabinoid or other ligand to the GPR55 receptor, comprising contacting the GPR55 receptor or functional fragment or variant thereof with a cannabinoid or other ligand in the presence or absence of a test compound, and comparing the amount of binding between the cannabinoid or other ligand and the GPR55 receptor or functional fragment or variant thereof in the presence or absence of the test compound, where an alteration in the amount of binding between the cannabinoid or other ligand and the GPR55 receptor or functional fragment or variant thereof in the presence of the test compound indicates that the test compound modulates binding between the cannabinoid or other ligand and the GPR55 receptor.

27. A method of identifying a compound that binds the GPR55 receptor, comprising incubating a cell expressing GPR55 receptor or functional fragment or variant thereof with a cannabinoid or other ligand in the presence or absence of a compound, and detecting displacement of cannabinoid or other ligand binding to the GPR55 receptor or functional fragment or variant thereof in the presence of the compound, where displacement of said binding is indicative of a compound that binds the GPR55 receptor.

28. A method of determining if a test compound is an agonist, antagonist or inverse agonist of the GPR55 receptor comprising
a) incubating a cell expressing GPR55 or functional fragment or variant thereof with the test compound;
b) measuring a signal indicative of receptor activation; and
c) comparing the measurement in b) with a second measurement of a signal indicative of receptor activation obtained from incubations performed in the absence of the test compound, where the test compound is an agonist of GPR55 if the signal indicative of receptor activation is higher in the presence of the test compound than in its absence, and wherein the test compound is an antagonist of GPR55 if the signal indicative of receptor activation is lower in the presence of the test compound than in its absence.

29. The method of claim 28, wherein the signal indicative of receptor activation is selected from intracellular phospholipase C activity, phospholipase A activity, adenylyl cyclase activity, cAMP level, MAP kinase activity, GTPγS binding, and intracellular concentration of calcium.

30. (canceled)

31. The method of any one of claims 1 or 10, wherein the cannabinoid ligand is selected from the group consisting of: anandamide, virohdamine, noladin ether, 2-aracodonylglycerol, palmityolethanolamine, oleylethanolamine and Δ9-tetrahydrocannbinol.

32. A method for determining the selectivity of a test compound against a panel of cannabinoid receptors, comprising measuring the modulatory effect of a test compound against GPR55 or any variant or functional fragment thereof, and at least one other cannabinoid receptor.

33. The method according to claim 32, wherein the cannabinoid receptor other than GPR55 is selected from the group consisting of: CB1, CB1a, CB1b and CB2.

34. The method according to claim 32 or 33, further comprising generating a profile of the test compound.
